SEQUENCE LISTING (8 sequences)

SEQ ID NO: 1 - protein, 121 aa, Artificial Sequence [sequence omitted]

SEQ ID NO: 2 - protein, 5 aa, Artificial Sequence
  Description of Artificial Sequence Synthetic peptide
SYWMH

SEQ ID NO: 3 - protein, 17 aa, Artificial Sequence
  Description of Artificial Sequence Synthetic peptide
EIDPSESNTNYNQKFKG

SEQ ID NO: 4 - protein, 12 aa, Artificial Sequence
  Description of Artificial Sequence Synthetic peptide
GGYDGWDYAIDY

SEQ ID NO: 5 - protein, 112 aa, Artificial Sequence [sequence omitted]

SEQ ID NO: 6 - protein, 16 aa, Artificial Sequence
  Description of Artificial Sequence Synthetic peptide
RSSQSLAKSYGNTYLS

SEQ ID NO: 7 - protein, 7 aa, Artificial Sequence
  Description of Artificial Sequence Synthetic peptide
GISNRFS

SEQ ID NO: 8 - protein, 9 aa, Artificial Sequence
  Description of Artificial Sequence Synthetic peptide
LQGTHQPYT

(19) United States
(12) Patent Application Publication
(10) Pub. No.: US 2023/0142437 A1
(43) Pub. Date: May 11, 2023

(54) DEVICES AND METHODS FOR TREATING A FISTULA

(71) Applicant: Millennium Pharmaceuticals, Inc., Cambridge, MA (US)

(72) Inventors: Vincent Ling, Walpole, MA (US); Sachiko Imaichi, Brookline, MA (US); Matthew Phaneuf, Ashland, MA (US); Nathaniel Long, Hudson, MA (US); Rayan Kassab, Taunton, MA (US); Daniel Leffler, Newton, MA (US)

(21) Appl. No.: 17/906,438

(22) PCT Filed: Mar. 18, 2021
(86) PCT No.: PCT/US2021/023022

§ 371 Date: Sep. 15, 2022

Related U.S. Application Data
(60) Provisional application No. 62/991,384, filed on Mar. 18, 2020.

(51) Int. Cl. A61B 17/00

(52) U.S. Cl. CPC A61B 17/0057; A61B 2017/00641

(57) ABSTRACT

Described herein are devices used for treating a fistula, along with methods of treatment thereof, and methods of manufacturing the device.

### RELATED APPLICATIONS

This application claims priority to U.S. Provisional Application No. 62/991,384, filed Mar. 18, 2020. The entire contents of the foregoing priority application are incorporated by reference herein.

### SEQUENCE LISTING

The instant application contains a Sequence Listing which has been submitted electronically in ASCII format and is hereby incorporated by reference in its entirety. Said ASCII copy, created on Mar. 18, 2021, is named T103022_1250 WO_0460_0_SL.txt and is 4,014 bytes in size.

### FIELD OF INVENTION

This disclosure relates to devices for implantation within a fistula tract to promote healing thereof, along with methods of manufacturing the device and using the device.

### BACKGROUND

Fistulas, such as perianal, rectovaginal, and enterocutaneous fistulas, are a common disorder. Generally, a fistula is an abnormal connection between two parts (e.g., tissues or structures) in the body of a subject, such as, for example, between the bowel and the vagina. The fistula typically has a primary opening (typically located within the body) and a tract or tunnel extending therefrom to a secondary opening. The tract can be relatively straight or follow a circuitous route. In some cases, the fistula may have multiple tracts. Common causes of fistula include injury, infection, surgical complications, congenital defect, and disease (e.g., Crohn's disease or ulcerative colitis). Treatments will vary depending on the cause, location, or nature of the fistula; however, fistulas can be difficult to treat and can frequently return.

Treatments typically include surgery, with or without the use of antibiotics, and can include, for example, placement of a seton for drainage, a flap procedure, or plugging a portion of the fistula (e.g., with fibrin glue). However, there are many side effects to surgical treatment, such as incontinence, reinfection, or recurrence of the fistula, along with a lengthy recovery time. Additionally, fistulas can take a very long time to heal on their own, if at all, creating additional hardship for the patient. There are also some so-called plugs on the market; however, these “plugs” typically include multiple components that need to be sutured together, and include biodegradable materials, which lead to high failure rates for example, ineffective fistula closure, detachment of the plug, or infection.

Accordingly, there is a need for improved devices and methods for promoting the closure of fistulas, with minimal side effects, reasonable treatment times, and reduced patient hardship.

### SUMMARY

Disclosed herein are non-biodegradable devices for plugging a fistula, where the devices promote faster healing, tissue integration, and optionally provide for the delivery of therapeutic agents. These devices are configured to resist dislocation from the implantation site. Also disclosed are methods of manufacturing the devices, methods of treating a patient with such devices, and kits containing such devices.

In one aspect, the invention relates to a device for implantation into a fistula tract within a subject or patient, wherein the fistula tract has a primary opening in a wall of a bodily structure and a tract extending from the primary opening. The device includes a unitary body configured to fill at least a portion of the fistula tract. The body includes an enlarged portion (also referred to herein as a head) located at a first end of the body and a tube-like structure extending from an area proximate the enlarged portion and terminating at a second end of the body, where the enlarged portion has a cross-sectional dimension equal to or greater than a cross-sectional dimension of the tube-like structure relative to a longitudinal axis of the body. In some embodiments, the device includes a neck located between the enlarged portion and the tube-like structure and having a reduced cross-sectional dimension relative to the cross-sectional dimensions of the enlarged portion and the tube-like structure. Generally, the device can be configured to provide an optimal fit in varying tract sizes and geometries and improved handling thereof.

Suitable fistulas include, but are not limited to, a perianal fistula, an enterocutaneous fistula or a rectovaginal fistula.

In various embodiments, at least a portion of the tube-like structure can be split into a plurality of elongate members. In other embodiments, the tube-like structure can be maintained as a unitary tubular shape, either in solid form or substantially hollow. Where the tube-like structure is split, the body can have anywhere from 1 to 20 elongate members (e.g., 1, 2, 3, 4, 5, 6, 7, 8, 9, 10, 11, 12, 13, 14, 15, 16, 17, 18, 19 or 20 elongate members). The number, size, and shape of the elongate members can vary to suit a particular application, for example, to accommodate the size and/or location of the fistula.

In some cases, one or more of the elongate members can be removed or otherwise modified (e.g., shortened) to suit the application or utilize a particular manufacturing process. For example, in some cases, it may be more efficient to manufacture the device with four (4) elongate members of equal length, while three (3) elongate members may be preferred for placement of the device within a body. In addition, in some cases it may be desirable for the device to have elongate members of different lengths, which can be obtained via a post-manufacturing (or pre-insertion) process. In various embodiments, the elongate members can be offset relative to a longitudinal axis of the device (e.g., the members flare outwardly from a common point). Additionally, the elongate members can be planar or have a variety of cross-sectional shapes, such as rectangular, trapezoidal, square, hexagonal, arcuate, oval, circular, etc.

In additional embodiments, at least a portion of the tube-like structure can be tapered towards the second end of the body. The enlarged portion can have a substantially planar profile and/or a substantially circular cross-section. The second end of the device can be configured for insertion into the primary opening with the first end configured to fill all or a portion of the fistula tract. In some cases, the first end of the device (e.g., the enlarged portion or head) is configured to fill the primary opening.

In some embodiments, the body can comprise one or more nanofibrous polymers, as described herein. In still other embodiments, at least a portion of the body includes an electrospun polymer, such as one or more nanofibrous synthetic materials, such as polyesters (e.g., polyethylene terephthalate (PET), polybutylene terephthalate (PBT), polytrimethylene terephthalate (PTT) and combinations thereof) and polyurethane (e.g., polycarbonate, polyether, polyester-based or combinations thereof) along with combinations of polyester and polyurethane. Generally, any biocompatible polymer (natural or synthetic) that can be subjected to electro-spinning may be used. In various embodiments, the nanofibrous polymer has a net charge throughout, within, or on a surface thereof. In some cases, the nanofibrous polymer is negatively charged. This nanofibrous polymer can include a polymer that has been modified prior to or post-electrospinning to contain a negative charge, e.g., by treatment with liquid sodium hydroxide (HYD) or a polyurethane polymer with a negative charge. In some cases, the nanofibrous polymer is positively charged. This nanofibrous polymer can include a polyester polymer that has been modified prior to or post-electrospinning to contain a positive charge, e.g., by treatment with liquid ethylenediamine (EDA).

Additionally, the device can include an electrospun material that has a therapeutic agent incorporated into the nanofibers. The therapeutic agent may include a small molecule. In some embodiments, the therapeutic agent is an antimicrobial agent, such as at least one of a bactericidal antibiotic, such as penicillin (e.g., ampicillin or amoxicillin), an aminoglycoside, ofloxacin; a bacteriostatic antibiotic, such as erythromycin, tetracycline, chloramphenicol; a quinolone antibiotic, such as ciprofloxacin; or a nitroimidazole, or a combination thereof. Additional antibiotic agents may include moxifloxacin, levofloxacin, linezolid, gentamycin, tobramycin, streptomycin, nafcillin, and doxycycline. The therapeutic agent may also include an anti-inflammatory agent, such as a NSAID, an IMSAID, an antileukotrienes, an anti-histamine (e.g., diphenhydramine), steroids (e.g., dexamethasone), or an immunosuppressive agent, such as azathioprine, tacrolimus, sirolimus, paclitaxel, everolimus, or biolimus. In further embodiments, the therapeutic agent may include an analgesic agent, such as a NSAID, acetaminophen, bupivacaine, meloxicam, or an opioid. In certain embodiments, the device can also include cells, such as, for example, allogeneic stem cells, autogenic stem cells, or mesenchymal stem cells (MSCs). The device may also include allogenic adipose tissue-derived stromal stem cells. Additional therapeutic agents include antifungal agents, such as diflucan, fluconazole, and itraconazole, anti-proliferate agents, such as paclitaxel, everolimus, and sodium butyrate; genetic agents, such as siRNA; growth factor agents, such as VEGF165, bFGF, and BMP-7; anticoagulant agents, such as recombinant hirudin/refludan, argatroban, and bivalirudan; material imaging agents, such as ditrizoic acid, barium sulfate, and gadodiamide; and various additives, such as thrombin, triclosan, nanosilver, lactoferrin, heterobifunctional cross-linkers, homobifunctional cross-linkers, lysozyme, activated proteins (activated drotrecogin alfa), ulex europaeus lectin I (UEA-1), and Galanthus nivalis lectin (GNL).

In another aspect, the invention relates to a device for implantation into a tract within the body, such as a perianal fistula, an enterocutaneous fistula or a rectovaginal fistula, that has a primary opening in a wall of a bodily structure and a tract extending from the primary opening. The device includes a body configured to fill at least a portion of the fistula tract and at least partially formed from an electrospun polymer. The body includes an enlarged portion located at a first end of the body and a tube-like structure extending from the enlarged portion and terminating at a second end of the body. The enlarged portion has a cross-sectional dimension equal to or greater than a cross-sectional dimension of the tube-like structure relative to a longitudinal axis of the body.

In various embodiments, the electrospun polymer comprises one or more nanofibrous synthetic materials, such as polyesters (e.g., polyethylene terephthalate (PET), polybutylene terephthalate (PBT), polytrimethylene terephthalate (PTT) and combinations thereof) and polyurethane (e.g., polycarbonate, polyether, polyester-based or combinations thereof) along with combinations of polyester and polyurethane. In some embodiments, the device comprises a mixture of nanofibrous polyethylene terephthalate and nanofibrous polybutylene terephthalate (nPET-PBT). In other embodiments, the device comprises nanofibrous polyurethane (nPU). In various embodiments, the nanofibrous polymer has a net charge throughout, within, or on a surface thereof. In some cases, the nanofibrous polymer is negatively charged. This nanofibrous polymer can include a polyester polymer that has been modified prior to or post-electrospinning to contain a negative charge (HYD) or a polyurethane polymer with a negative charge. In some cases, the nanofibrous polymer is positively charged. This nanofibrous polymer can include a polyester polymer that has been modified prior to or post-electrospinning to contain a positive charge (EDA). The device can be non-biodegradable.

In additional embodiments, the device includes an electrospun material that has a therapeutic agent incorporated into the nanofibers. The therapeutic agent may include a small molecule. In some embodiments, the therapeutic agent is an antimicrobial agent, such as at least one of a bactericidal antibiotic, such as penicillin (e.g., ampicillin or amoxicillin), an aminoglycoside, ofloxacin; a bacteriostatic antibiotic, such as erythromycin, tetracycline, chloramphenicol; a quinolone antibiotic, such as ciprofloxacin; or a nitroimidazole, or a combination thereof. The therapeutic agent may also include an anti-inflammatory agent, such as a NSAID, an IMSAID, an antileukotrienes, steroids (e.g., dexamethasone), or an immunosuppressive agent, such as azathioprine, tacrolimus, sirolimus, paclitaxel, everolimus, or biolimus. In further embodiments, the therapeutic agent may include an analgesic agent, such as a NSAID, acetaminophen, bupivacaine, meloxicam, or an opioid. In certain embodiments, the device can also include cells, such as, for example, allogeneic stem cells, autogenic stem cells, or mesenchymal stem cells (MSCs). The device may also include allogenic adipose tissue-derived stromal stem cell.

In certain embodiments, the body is a unitary structure as described herein. The body can also include a neck portion located between the enlarged portion and the tube-like structure, where the neck has a reduced cross-sectional dimension relative to the cross-sectional dimensions of the enlarged portion and the tube-like structure. In some embodiments, at least a portion of the tube-like structure can be tapered towards the second end of the body and/or at least a portion of the tube-like structure can be split into a plurality of elongate members as described herein. For example, the plurality of elongate members can be offset relative to the longitudinal axis and can include from 1 to 20 elongate members. In various embodiments, the enlarged portion can have a substantially planar profile or a substantially circular cross-section. The second end of the device can be configured for insertion into the primary opening with the first end configured to fill all or a portion of the fistula tract. In some cases, the first end of the device is configured to fill the primary opening.

In yet another aspect, the invention relates to a method of manufacturing a device for implantation into a tract within the body, such as a perianal fistula, an enterocutaneous fistula or a rectovaginal fistula which has a primary opening in a wall of a bodily structure and a tract extending from the primary opening. The method includes the steps of preparing a polymeric solution, loading the solution into an electro-spinning unit, perfusing the polymeric solution at a defined rate into an injection needle port that has a defined voltage applied to the needle, collecting the resulting nanofibers emanating from the needle port onto a rotating collector in the form of a mandrel having a fluted portion to create a unitary tube-like electro-spun body with a fluted portion, performing a post-treatment process, such as vacuum heating or solvent extraction, to remove residual solvent; and removing the electrospun body from the mandrel. The method further includes manipulating the fluted portion by pulling the fluted end downward to form an enlarged portion relative to the rest of the body, where the enlarged portion has a cross-sectional dimension larger than a cross-sectional dimension of the rest of the body; flattening the rest of the body to form a substantially planar structure extending from the elongate portion; splitting the planar structure along a longitudinal axis of the body to form a plurality of elongate members; and forming a neck region between the enlarged portion and the elongate members by sealing a portion of the tube-like body proximate to the enlarged portion, where the neck region has a cross-sectional dimension less that the cross-sectional dimensions of the enlarged portion and the rest of the body.

In various embodiments, the step of preparing the solution includes weighing polyester and/or polyurethane chips and placing them into a borosilicate vial, adding hexafluoroisoproposal (HFIP) to chips within the vial, sealing the vial containing the prepared solution, and agitating the vial until all pellets in solution are dissolved. In some embodiments, the weighing step includes weighing polyethylene terephthalate (PET) and polybutylene terephthalate (PBT) chips. In some embodiments, the polymeric solution can have a therapeutic agent, such as those disclosed herein, added into the solution prior to electrospinning.

The step of removing the electrospun body from the mandrel includes rinsing the body with, for example, 100% ethanol (EtOH) over a sink, or other receptacle; filling a non-pyrogenic serological tube or similar device with 100% ethanol; inserting the body and mandrel within the tube; placing the tube with the mandrel into a 250 mL graduated cylinder filled with deionized water (DIH₂O); placing the cylinder within a sonicator; sonicating the cylinder; removing the mandrel from the tube; rinsing the body with DIH₂O water; and drying the body.

In some embodiments, the fluted portion of the mandrel is substantially centrally located along a length of the mandrel, which can enable the production of two bodies from a single mandrel. For example, the step of removing the electrospun body from the mandrel can include splitting the body (e.g., cut with a razor or similar instrument) substantially perpendicularly to a longitudinal axis of the body at a central region of the fluted portion to create two bodies, each having a fluted end. In various embodiments, the size and shape of the mandrel can vary to suit a particular application, such as producing bodies of differing lengths and diameters or producing more than two bodies on a single mandrel. Additionally, the body can be subjected to a surface modification, such as an anionic or cationic surface modification. The step of forming the neck region can include ultrasonically welding a portion of the tube-like body proximate to the enlarged portion.

In still another aspect, the invention relates to kits including one or more of the devices for implantation in a tract as described herein. In some embodiments, the kit may include several devices of varying dimensions or materials, with an optimum device chosen by medical personnel at the time of implantation. The kits may also include a non-biodegradable suture, such as one made from an electrospun material, and/or one or more surgical instruments configured to assist with, for example, implantation.

In still yet another aspect, the invention relates to a method of treating a patient having a fistula that has a primary opening in the wall of a bodily structure, and a fistula tract extending from the primary opening. The method includes the insertion of any one (or more) of the devices described herein. The device can be inserted into the fistula tract through the primary opening, where the device can fill at least a portion of the fistula tract.

In various embodiments, the patient has a perianal fistula, such as a cryptoglandular anal fistula. In some embodiments, the patient has a rectovaginal fistula, an enterocutaneous fistula, or an enteroatmospheric fistula. In some cases, the patient may have Crohn's disease, ulcerative colitis, chronic diarrhea, tract formation post-pregnancy, or rectal cancer. The method can include inserting the device into the fistula tract. In certain embodiments of the method, the device is maintained in the fistula tract for a time sufficient for the fistula to heal. The device can promote or permit ingrowth of tissue into the device. For example, the device can alter cellular dynamics at the site of insertion. Additionally, the device does not induce fibrosis at the site of insertion, and/or does not induce collagen deposition at the site of insertion.

Still other aspects, embodiments, and advantages of these exemplary aspects and embodiments, are discussed in detail below. Moreover, it is to be understood that both the foregoing information and the following detailed description are merely illustrative examples of various aspects and embodiments, and are intended to provide an overview or framework for understanding the nature and character of the claimed aspects and embodiments. Accordingly, these and other objects, along with advantages and features of the present invention herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention and are not intended as a definition of the limits of the invention. For purposes of clarity, not every component may be labeled in every drawing. In the following description, various embodiments of the present invention are described with reference to the following drawings, in which:

FIG. 1A is a pictorial representation of a fistula plug in accordance with one or more embodiments of the invention;

FIG. 1B is an enlarged pictorial representation of the material of a fistula plug in accordance with one or more embodiments of the invention;

FIG. 2 is a pictorial representation of an alternative fistula plug in accordance with one or more embodiments of the invention;

FIG. 3 is a flow chart depicting the various steps for manufacturing a fistula plug in accordance with one or more embodiments of the invention;

FIGS. 4A-4I are pictorial representations of certain exemplary process steps as may be practiced in accordance with one or more embodiments of the invention;

FIGS. 5A-5C are pictorial representations of surface modifications of a fistula plug in accordance with one or more embodiments of the invention;

FIGS. 6A and 6B are pictorial representations of applications for the devices disclosed herein in accordance with one or more embodiments of the invention;

FIG. 7 is a graphical representation of cell attachment times for plugs made of different materials in accordance with one or more embodiments of the invention;

FIGS. 8A-8C are pictorial representations of mesenchymal stem cells growing on control, EDA (positive), and HYD (negative) nPET-PBT nanofibrous plug materials in accordance with one or more embodiments of the invention;

FIG. 8D is a pictorial representation of a nanofibrous plug material before cell seeding for reference;

FIG. 9 is a pictorial representation of a non-drug loaded plug versus two drug loaded plugs representing the effectiveness of drug loading in accordance with one or more embodiments of the invention;

FIG. 10 is a collection of photographs depicting the differences in treating with and without implantation of a plug in accordance with one or more embodiments of the invention;

FIG. 11 is a collection of photographs depicting a plug that has been subjected to staining after implantation in accordance with one or more embodiments of the invention;

FIG. 12A is a collection of photographs depicting the implantation of a plug in accordance with one or more embodiments of the invention;

FIG. 12B is a collection of photographs depicting the implantation site of the plug in FIG. 12A after 30 days in accordance with one or more embodiments of the invention;

FIG. 13 is a collection of microscopic images for comparison following the implantation in a fistula tract of a plug in accordance with one or more embodiments of the invention (bottom panel), a plug fabricated from a different (non-nanofibrous) plug material (upper right panel), and a control tract without implantation of a plug device (upper left panel); and

FIGS. 14 and 15 are microscopic images of an implanted plug after staining in accordance with one or more embodiments of the invention.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

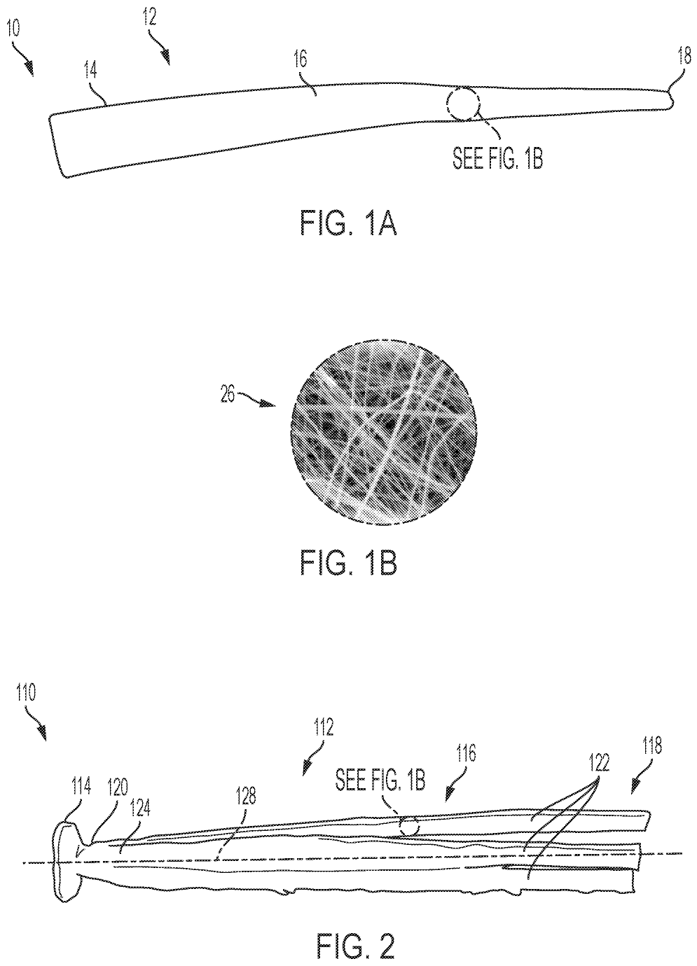

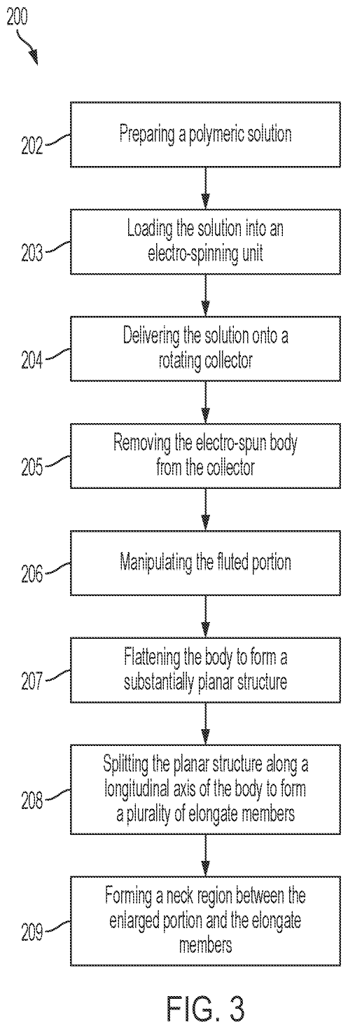

FIG. 1 depicts one embodiment of a fistula plug device 10 having an overall club-like shape or configuration. The plug 10 is made up of a body 12 having a first, enlarged end 14 and a tube-like structure 16 extending approximately from the enlarged end 14 to a second end 18 of the body 12. As can be seen, the tube structure 16 tapers towards the second end 18 so as to facilitate implantation of the plug 10. Generally, the plugs disclosed herein are configured to be inserted through a primary opening in a wall of a bodily structure (e.g., via the tapered end) and reside within at least a portion of a fistula tract extending from the primary opening.

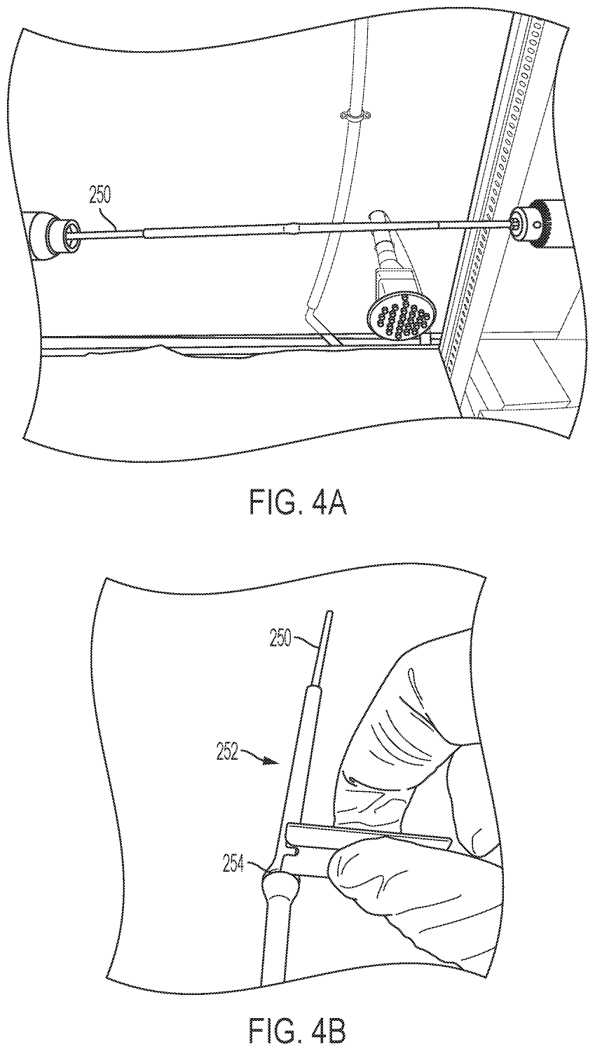

FIG. 2 depicts an alternative embodiment of a fistula plug device 110 having a “jelly” configuration. Similar to the plug 10 of FIG. 1, the plug 110 includes an enlarged head 114 disposed at a first end of the body 112 and a tube-like structure 116 extending therefrom towards a second end 118 of the body 112. In some embodiments, the plug can optionally comprise a “neck” region 120 positioned between the enlarged head 114 and the tube like structure 116. Neck region 120 can comprise a reduced cross-sectional dimension relative to the cross-sectional dimensions of the enlarged head and the tube-like structure. In the embodiment shown in FIG. 2, the tube-like structure has been segmented into a plurality of elongate members 122, which generally extend from a common point 124 proximate to the head 114 and neck 120 and flare outwardly from a longitudinal axis 128 of the device 110. However, the common point from which the elongate members extend can vary to suit a particular application resulting in, for example, a device with a body partially made of a tubular structure and partially made of elongate members. In some embodiments, the elongate members are formed by segmenting tube-like structure 116 into two or more elongate members, e.g., by slicing or cutting. Typically, in devices that comprise a neck region, the neck 120 is disposed between the head 114 and the elongate members 122, with the neck 120 providing greater strength and/or stability to the overall plug. In other embodiments, elongate members 122 extend directly from the head 114. Generally, the tube-like structure 116 (with or without elongate members) will be configured to fill all or a portion of a fistula tract.

Four roughly planar elongate members 122 with substantially equal widths and lengths are generally shown in FIG. 2; however, the number of members 122 can vary, along with the size and shape of each elongate member 122 (e.g., the lengths of the members can vary between members and the cross-sectional shapes can include rectangular, trapezoidal, square, hexagonal, oval, circular, etc.). In some embodiments, the device comprises 2 or more elongate members, e.g., 2 or more, 3 or more, 4 or more, 5 or more, 6 or more, 7 or more, 8 or more, 9 or more, 10 or more, 11 or more, or 12 or more, 15 or more, 20 or more, or 25 or more elongate members. For example, in some embodiments, the device comprises 2-25 elongate members. In some embodiments, the device comprises 2-15 elongate members. In some embodiments, the device comprises 2-10 elongate members. In some embodiments, the device comprises 1-4 elongate members. In some embodiments, the device comprises 4-8 elongate members. In some embodiments, the device comprises 8-12 elongate members. In some embodiments, the device comprises 10-20 elongate members.

The head 14, 114 is shown in FIG. 1 and FIG. 2 as substantially circular with a planar profile; however, the shape and size of the head can vary to suit a particular application (e.g., as necessary to fill a primary opening of a fistula). The overall size and shape of the devices 10, 110 can also vary to suit a particular application (e.g., location of the fistula, size of the fistula, type of fistula, etc.). For example, enterocutaneous fistulas may have wider openings to close than a perianal fistula, so the plug should be wide enough and long enough to appropriately seal the fistula. In various embodiments, the first or enlarged end 14, 114 of the device 10, 110 can have a diameter (e.g., an outer dimension generally) of about 1 mm to about 10 cm (average diameter 2-4 mm), a length of about 1 cm to about 30 cm (average length of 3-5 cm), and a wall thickness of about 0.050 mm to 10 cm (average wall thickness (0.5 mm-1 mm).

In some embodiments, the enlarged end 14, 114 can have a cross-sectional diameter of about 1 mm to about 10 cm. In some embodiments, the enlarged end 14, 114 can have a cross-sectional diameter of about 10 mm to about 1 cm. In some embodiments, the enlarged end 14, 114 can have a cross-sectional diameter of about 50 mm to about 2 cm. In some embodiments, the enlarged end 14, 114 can have a cross-sectional diameter of about 1 cm to about 3 cm. In some embodiments, the enlarged end 14, 114 can have a cross-sectional diameter of about 3 cm to about 5 cm. In exemplary embodiments, the enlarged end 14, 114 can have a cross-sectional diameter of about 1 mm, 5, mm, 10 mm, 20 mm, 30 mm, 40 mm, 50 mm, 60 mm, 70 mm, 80 mm, 90 mm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, or 10 cm.

In some embodiments, the fistula plug device can have a length of about 1 cm to about 30 cm. In some embodiments, the fistula plug device can have a length of about 1 cm to about 3 cm. In some embodiments, the fistula plug device can have a length of about 1 cm to about 5 cm. In some embodiments, the fistula plug device can have a length of about 3 cm to about 8 cm. In some embodiments, the fistula plug device can have a length of about 5 cm to about 10 cm. In some embodiments, the fistula plug device can have a length of about 8 cm to about 12 cm. In some embodiments, the fistula plug device can have a length of about 10 cm to about 20 cm. In some embodiments, the fistula plug device can have a length of about 20 cm to about 30 cm. In exemplary embodiments, the fistula plug device can have a length of about 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, 26 cm, 27 cm, 28 cm, 29 cm, or 30 cm.

In some embodiments, the fistula plug device can have a wall thickness of about 1 mm to about 10 cm. In some embodiments, the fistula plug device can have a wall thickness of about 10 mm to about 1 cm. In some embodiments, the fistula plug device can have a wall thickness of about 50 mm to about 2 cm. In some embodiments, the fistula plug device can have a wall thickness of about 1 cm to about 3 cm. In some embodiments, the fistula plug device can have a wall thickness of about 3 cm to about 5 cm. In exemplary embodiments, the fistula plug device can have a wall thickness of about 1 mm, 5, mm, 10 mm, 20 mm, 30 mm, 40 mm, 50 mm, 60 mm, 70 mm, 80 mm, 90 mm, 1 cm, 1.5 cm, 2 cm, 2.5 cm, 3 cm, 3.5 cm, 4 cm, 4.5 cm, 5 cm, 5.5 cm, 6 cm, 6.5 cm, 7 cm, 7.5 cm, 8 cm, 8.5 cm, 9 cm, 9.5 cm, or 10 cm.

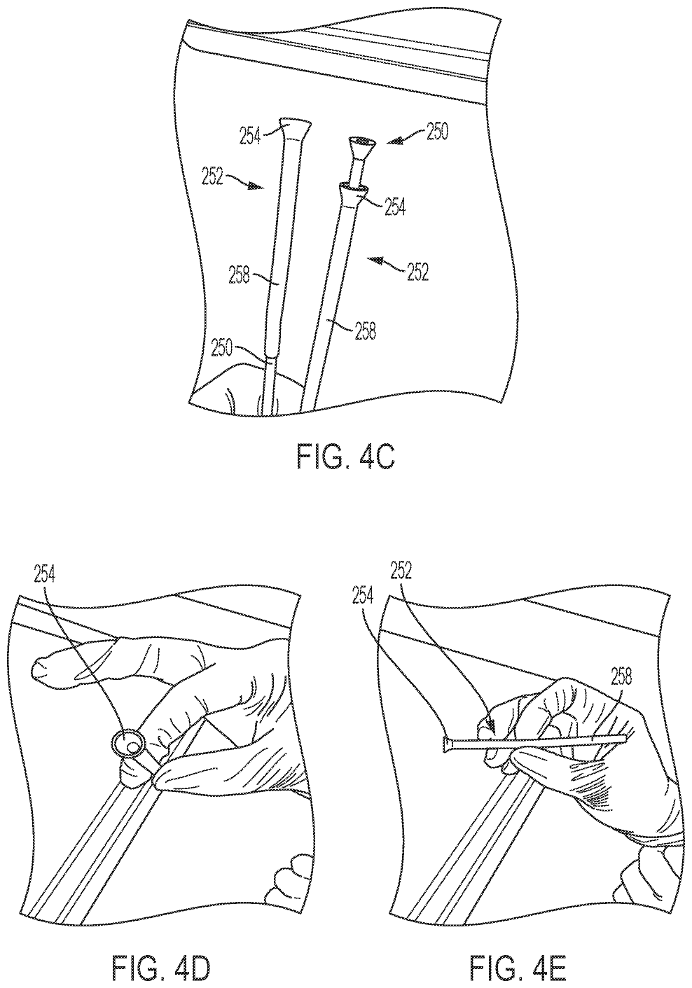

The devices can be formed as a unitary body that is fabricated as a single piece. Fistula plugs having a unitary body design can be advantageous in relation to plugs fabricated as two or more separate pieces that are joined together prior to administration, at least because the two or more pieces can undesirably become separated following implantation in a subject. In contrast, fistula plugs formed as a single unitary body do not have multiple pieces that can separate following implantation. An exemplary process for producing a fistula plug device is described below with respect to FIG. 3.

In some embodiments, the fistula plug devices described herein are fabricated from nanofibrous polymers. In some embodiments, the nanofibrous polymers are prepared by electrospinning. An exemplary process for producing a fistula plug device comprising electrospun polymer material is described in FIG. 3.

As shown in FIG. 1B, the electrospun material 26 typically has a spider web-like quality to it that resembles an extracellular scaffold that may be found in nature. Such a configuration promotes tissue integration, prevents rejection, and/or provides a structure for introducing a therapeutic agent or cellular material to the fistula to promote healing when implanted. Electrospun polymers contain spaces or openings between the overlapping fibers. In some embodiments, the openings in the scaffold (i.e., between the fibers of the scaffold) can range from about 0.1 μm to about 100 μm, e.g., 0.1 μm to about 10 μm; 10 μm to about 20 μm; 20 μm to about 50 μm; 50 μm to about 80 μm; or 80 μm to about 100 μm. These dimensions are exemplary only and the specific sizes and shapes can be adjusted to suit a particular application.

The fistula plug devices described herein can be fabricated from any suitable material. In some embodiments, the fistula plug can be fabricated from a non-biodegradable polymer. Exemplary non-biodegradable polymers include, but are not limited to, polyethylene terephthalate (PET, also known as Dacron), polybutylene terephthalate (PBT), polytrimethylene terephthalate (PTT), polyurethane (PU), polytetrafluoroethylene (ePTFE), poly(glycolic acid) (PGA), gelatin, or alginate. In some embodiments, the polymer is a nanofibrous polymer. In some embodiments, the polymer is a nanofibrous electrospun polymer. In some embodiments, the fistula plug device comprises a single type of polymer. In other embodiments, the fistula plug device can comprise more than one type of polymer. In some embodiments, the fistula plug device comprises a mixture of 2 or more, 3 or more, 4 or more, or 5 or more types of polymer. In some embodiments, the fistula plug device comprises a mixture of 2, 3, 4, 5, 6, 7, 8, 9, or 10 or more types of polymer. In exemplary embodiments, the fistula plug device comprises nanofibrous polyethylene terephthalate (nPET). In other exemplary embodiments, the fistula plug device comprises nanofibrous polybutylene terephthalate (PBT). In other exemplary embodiments, the fistula plug device comprises a mixture of nanofibrous polyethylene terephthalate and nanofibrous polybutylene terephthalate (nPET-PBT). In other exemplary embodiments, the fistula plug device comprises nanofibrous polyurethane (nPU). In some embodiments, the fistula plug device comprises one or more nanofibrous polymer, wherein at least one of the nanofibrous polymers is selected from the group consisting of nPET, nPBT, nPU, and combinations thereof. Other polymers are contemplated and considered within the scope of the invention, particularly such polymers as are suitable for fabrication of the device using electrospinning.

Generally, any biocompatible polymers suitable for use in electrospinning can be used in the device described herein. Other polymers suitable for use in the scaffold layers of the device described herein include, but are not limited to poly-lactic acid (PLA), poly-glycolic acid (PGA), poly-lactic-co-glycolic acid (PLGA), polycaprolactone (PCL), polypropylene (PP), and poly-tetrafluoroethylene (PTFE), polyvinyl alcohol (PVA), polyethylene oxide (PEO), polytrimethylene terephthalate (PTT), polyethylene acetate (PEVA), poly-D-lactide (PLDA), polylactic acid (PLLA), or polyethylene glycol (PEG). Other suitable polymers include, but are not limited to, collagen, gelatin, alginate, fibrinogen, silk, elastin, cellulose, chitin, chitosan.

In other embodiments, the fistula plug can be fabricated from a biodegradable polymer. Exemplary biodegradable polymer materials include, but are not limited to, collagen, cellulose, elastin, glycosaminoglycans, peptidoglycans, chitin, or fibrin. The biodegradable material can be optionally treated with a crosslinker, aldehydes (e.g., glutaraldehyde), carbodiimides, acrylamide, or diimidmates. In some embodiments, the biodegradable 1 material includes glutaraldehyde-cross linked biological membranes. In one embodiment, the fistula plug device can comprise a combination of one or more non-biodegradable polymers blended/combined with one or more biodegradable polymers. In some embodiments, the fistula plug device can be fabricated from hydrophilic polyurethanes, polycaprolactone (PCL), polylactide (PLA), poly(lactic-co-glycolic acid) (PLGA), polyglycolic acid (PGA), or alginate, or combinations thereof.

In some embodiments, the fistula plug device comprises the same polymer or mixture of polymers throughout the device. In other embodiments, the fistula plug device contains multiple polymers dispersed heterogeneously through the device. For example, the device can comprise a first polymer or mixture of polymers inside the device, and a second polymer or mixture of polymers on the external surface of the device (i.e., the surface of the device that will be in contact with the subject following implantation). In an exemplary embodiment, a fistula plug device can comprise an inner polymer comprising nPET-PBT, and an external polymer comprising nPU.

In various embodiments, all or a portion of the polymers present in the device can be modified to suit a particular application. For example, the nanofibrous polymer can have a net positive or negative charge disposed within or throughout the polymer or on a surface thereof. Substituents conferring a net charge to the polymer can be added to the polymer prior to, during, or after fabrication of the polymer into the fistula plug device. In certain embodiments, the external surface of the device comprises a positive or negative charge. A surface charge can be used to attract/repel cells, as certain cell types have an inherent charge on their surface receptors/proteins. A charged polymer can also attract specific proteins in the blood and microenvironment surrounding the device that promote cell attachment (e.g., glycoproteins, proteins with RGD sequences). Ionic surfaces can also improve surface wettability and contact with cells. In an exemplary embodiment, a polymer in the fistula plug device can be modified to include a net negative charge. A net negative charge can be imparted on a polymer in the fistula plug by, for example, contacting the plug (or a polymer thereof) with a reagent promoting alkaline hydrolysis, e.g., by treatment with liquid sodium hydroxide (HYD). In another exemplary embodiment, a polymer in the fistula plug device can be modified to include a net positive charge. A net positive charge can be imparted on a polymer in the fistula plug by, for example, contacting the plug (or a polymer thereof) with a diamine, such as ethylene diamine (EDA), 2-methylpentamethylene diamine, 1,2-diaminocyclohexane, or 1,6-hexanediamine Other examples of charged surface modifications and methods of generating charged surface modifications are further described, for example, in U.S. Pat. No. 6,743,253B2 and U.S. Pat. No. 7,037,527B2, which are hereby incorporated by reference in their entirety.

In some embodiments, the fistula plug device can be loaded with an agent, e.g., a therapeutic agent. For this process, a pre-set amount of the active agent or therapeutic agent is measured (typically by weight) and added into the polymer solution prior to electrospinning. In other embodiments, the agent can be loaded into the device after fabrication, e.g., by soaking the device in a solution or suspension containing the agent. In some embodiments, the therapeutic agent is a small molecule. In other embodiments, the therapeutic agent is a peptide or protein. In other embodiments, the therapeutic agent is a nucleic acid, e.g., DNA (including expression vector DNA, e.g., plasmid DNA, viral vector DNA, etc.), mRNA, miRNA, antisense RNA, siRNA, gRNA, etc. In other embodiments, the therapeutic agent is a cell.

In some embodiments, the fistula plug device can comprise one or more antimicrobial agents. The presence of an antimicrobial agent in the fistula plug can slow, reduce, or prevent the growth of microbes, e.g., bacteria, in the fistula tract. In some embodiments, the fistula plug device comprises a bactericidal antibiotic. In some embodiments, the fistula plug comprises a bacteriostatic antibiotic. In some embodiments, the fistula plug comprises a quinolone antibiotic. In exemplary embodiments, the fistula plug comprises one or more of the following: penicillin, an aminoglycoside, ofloxacin, erythromycin, tetracycline, chloramphenicol, ciprofloxacin, nitroimidazole, metronidazole, or a combination thereof. For example, the fistula plug device can comprise ciprofloxacin, metronidazole, or a combination of ciprofloxacin and metronidazole.

In some embodiments, the fistula plug device comprises one or more anti-inflammatory agents. The presence of an anti-inflammatory agent in the fistula plug can reduce or prevent inflammation in the fistula tract. For example, in some embodiments, the fistula plug can comprise a nonsteroidal anti-inflammatory drug (NSAID), an immune selective anti-inflammatory derivative (IMSAID), an antileukotriene, or a steroid (e.g., dexamethasone), or a combination thereof.

In some embodiments, the fistula plug device comprises one or more immunosuppressive agents. For example, in some embodiments, the fistula plug can comprise azathioprine, tacrolimus, sirolimus, paclitaxel, everolimus, or biolimus, or a combination thereof.

In some embodiments, the fistula plug device comprises one or more analgesic agents, such as a NSAID, acetaminophen, bupivacaine, meloxicam, or an opioid, or a combination thereof.

In some embodiments, the fistula plug device can comprise cells. Cells for incorporation into the fistula plug can be allogeneic or autologous. In some embodiments, the cells can be stem cells, e.g., allogeneic stem cells or autologous stem cells. In some embodiments, the cells are mesenchymal stem cells (MSCs). In other embodiments, the cells are adipose tissue-derived stromal stem cells (allogeneic or autologous). In such embodiments, the device can be loaded with cells prior to implantation in a subject, using known techniques. For example, the device can be incubated with cells in an in vitro cell culture system for a period of time sufficient for cells to adhere to the device.

In some embodiments, the fistula plug device can comprise combinations of any one or more (e.g., 1, 2, 3, 4, 5, 6, 7, 8, 9, 10 or more) of the foregoing agents. For example, in some embodiments, the fistula plug device can comprise one or more of an antimicrobial agent, an anti-inflammatory agent, an immunosuppressive agent, an analgesic agent, or a cell.

FIG. 3 is a flow chart 200 depicting the various steps that may be used in exemplary methods to create a fistula plug in accordance with one or more embodiments of the invention. Generally, the method is described in terms of a manual, small-scale batch process; however, the process can be scaled up and automated (e.g., via robotics) and run as a continuous process to suit large-scale production, as would be known to a person of ordinary skill in the field of industrial engineering.

The first step 202 involves preparing the solution for producing the body. Generally, a polymeric solution is prepared by weighing polymer chips (e.g., polyester and/or polyurethane chips) and placing them into a vial (e.g., a borosilicate vial). In some embodiments, (0.5%-30% w:v) polyethylene terephthalate (PET) and (0.5%-30% w:v) polybutylene terephthalate (PBT) chips are used. Next, a solvent (e.g., hexafluoroisoproposal (HFIP)) is added to the chips within the vial and sealed by, for example, screwing a cap onto the vial and sealing with a flexible film, such as, for example, Parafilm® as available from Bemis Company, Inc., headquartered in Neenah, Wis. The sealed vial is then agitated (e.g., by placement on a rotator set at a suitable speed, e.g., 45 RPM) until all of the chips within the solution are dissolved (about 5-7 days or more).

At step 203, the polymeric solution can now be introduced to an electro-spinning unit. In one embodiment, the unit can be loaded via a syringe. Specifically, a 10 mL syringe can be used by removing the plunger and connecting the end of the syringe to the needle port. Following introduction of the solution into the syringe, the syringe can be connected to a pump on the electro-spinning unit.

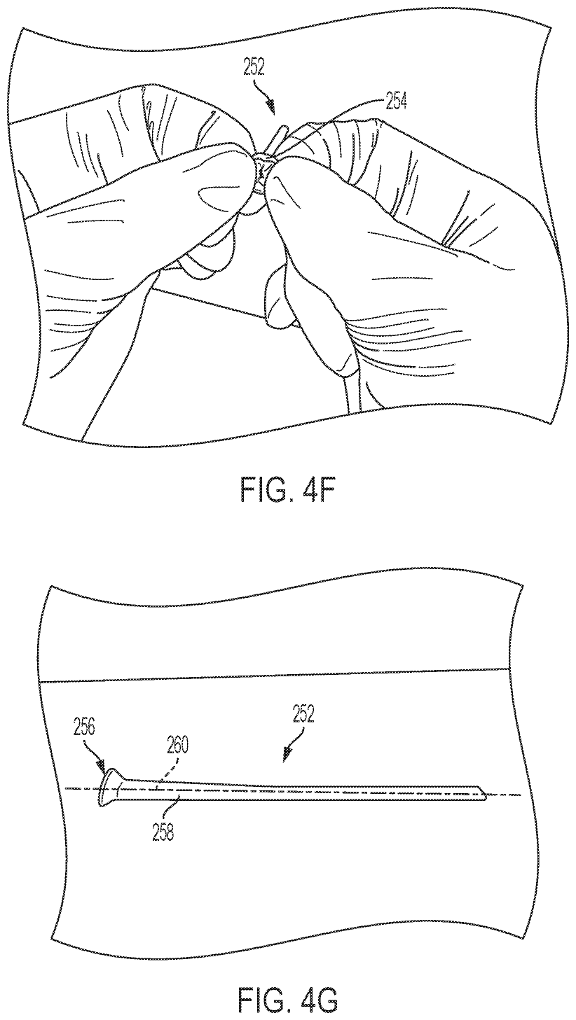

Moving to step 204, the solution will be delivered onto a rotating collector 250, such as a mandrel. Delivering the solution includes the steps of perfusing the polymeric solution at a defined rate into an injection needle port that has a defined voltage applied to the needle and collecting the resulting nanofibers emanating from the needle port onto a rotating collector in the form of a mandrel having a fluted portion to create a unitary tube-like electro-spun body with a fluted portion. See FIG. 4A. The operating parameters of the electrospinning unit can be determined to suit a particular application, such as the number and size of the devices to be made. In one embodiment, the e-spinning distance is set at about 10-20 cm, a rotation speed of about 50 to 150 RPM, and a voltage of about 20-23 kV. The mandrel size used and spin time will depend on what size and types (club vs. jelly) devices are to be made. Two examples of mandrel sizes and spin times are shown in Table 1 below. The mandrels are typically Teflon coated steel mandrels; however, other materials may be selected to suit a particular application. Additionally, the overall shape of the mandrel can vary to suit a particular application. In a particular embodiment, two mandrels, each with a fluted section connected in the middle of the collector, are used to produce two jelly type devices. In some embodiments, the mandrel is tapered along its length. Additionally, the mandrel diameters can run from about 1 mm to about 30 mm and have lengths of about 5 cm to about 60 cm.

TABLE 1

| Mandrel Size (diameter) | Spin Time |
|---|---|
| 2 mm | 15 minutes |
| 4 mm | 60 minutes |

At the end of the spin, any excess material can be removed by, for example, being cut off with a razor blade. In one embodiment, the razor blade is held against the mandrel and the mandrel is spun around to produce a clean cut of material, which can be removed and discarded. In addition, a post-treatment process can be performed on the electrospun body, such as vacuum heating or solvent extraction to remove residual solvent.

In some embodiments, a therapeutic agent, such as those disclosed herein, can be added to the polymeric solution prior to the electrospinning process, such that a therapeutic agent (e.g., an antimicrobial or anti-inflammatory) can be delivered to a patient when the device is implanted. In addition, the polymeric solution can be further modified prior to electrospinning to produce an electrospun body having a net charge (i.e., negatively or positively charged).

The step of removing the electrospun body 252 from the collector 250 (step 205) can begin with removing the collector being held in place and transferring same to a sonicator. The body 252 can be rinsed with 100% ethanol (EtOH) over a sink or other type of collection basin. Next, a non-pyrogenic serological tube (or similar, as may be designated in the lab) can be filled with 100% ethanol (e.g., enough to cover the body on the collector placed within the tube). The tube, with the collector inside, can be placed into a container (e.g., 250 mL graduated cylinder) filled with deionized water (DI-H₂O). The container can then be placed inside of a sonicator. The electrospun body should otherwise remain untouched. The sonicator can be run for about 30 minutes. After the 30-minute sonication in ethanol, the collector 250 can be removed from the serologic tube and rinsed with DI-H₂O. The EtOH can be emptied from the tube and, the tube can be filled with DI-H₂O. The collector can be placed back into the tube and both placed back inside of the container. The container can be placed back into the sonicator and sonicated for about 2 minutes. At the end of the 2-minute sonication, the collector can be removed from the tube and placed on a surface for drying at room temperature for approximately 24 hours. In some embodiments, the drying surface can include, for example, a paper-based substrate, such as cardboard, paperboard, fiberboard, etc. In some embodiments, mechanical means for drying the body may be used, such as, for example, a tunnel dryer, infrared heating, or other means of exposing the body to a flow of air.

After the material has dried, the body 252 can be removed from the collector 250 to form two fistula plug devices. Using a cutting instrument (e.g., a sharp razor blade), the body 252 can be cut about the axis proximate the mid-point of the collector and generally corresponding to the center of the fluted portion or bead 254. See FIG. 4B. Cut completely about the bead to separate the body into two pieces. The bodies 252 can be loosened on the mandrel by lightly twisting the material with a gloved hand. For the two-piece mandrel, take one side and twist one body and repeat for the other side. Once the bodies are completely loose and can spin around mandrel, they can be slid off of the mandrel (e.g., away from the fluted portion of the mandrel). See FIGS. 4C-4E.

Steps 206-209 are primarily directed to forming a jelly type of device as shown in FIG. 2. Prior to forming the final device, the body can be exposed to a surface treatment, such as either an anionic or a cationic surface modification. Generally, the surface modification process can be carried out at essentially any stage of the manufacturing process to suit a particular application.

Surface modifications can be added to the polymer in accordance with art recognized methods, before, during, or following fabrication of the plug. For example, to create an anionic surface modification, the body can be inserted onto a 4 mm Teflon coated mandrel. The mandrel with body can then be placed into a 1000 mL beaker of boiling water containing NaOH, e.g., 0.5% weight to volume of NaOH, for about 30 minutes. In some embodiments, at least about 6 cm of the body can be in contact with the solution. After boiling for about 30 minutes, the mandrel and body can be removed from the solution, rinsed with DI-H₂O for about 30 seconds, and allowed to air dry at room temperature.

In another embodiment, to create a cationic surface modification, the following exemplary procedure can be followed. The body can be inserted onto a 4 mm Teflon coated mandrel. The mandrel with body can be placed into a 600 mL beaker containing about 250 mL of 50% ethylenediamine (EDA). The body can be allowed to soak in the solution for about 4 hours. After the treatment is over, the mandrel and body can be removed from the solution, rinsed with DIH₂O, and fully submerged in DI-H₂O for 24 hours. After 24 hours has ended, the body can be removed from the DI-H₂O and allowed to dry at room temperature.

Step 206 is directed to forming the enlarged portion of the body, which generally involves manipulating the fluted portion 254 of the body 252. See FIG. 4F. The process requires taking the material of the fluted portion and lightly pulling down the cone shaped area to form the head. The material does not need to be stretched, but only pulled in order to form a substantially circular, flat head 256. It should be noted that other shapes and configurations of the head are contemplated and considered within the scope of the invention. It should also be noted that the tube-like structure 258 extending from the head 256 can be tapered and the body formed at this point could be used as a club type device.

Next, the body 252 can be placed on a solid surface (e.g., a cutting board or robotic platform) and the tube-like structure 258 that extends from the enlarged head portion 256 can be flattened (step 207). The body is now configured with an enlarged head portion and a relatively planar body extending therefrom. See FIG. 4G.

Step 208 is generally directed towards splitting the planar body along its longitudinal axis 260 into a plurality of elongate members 262. See FIG. 4H. The number of elongate members will vary to suit a particular application. Using a cutting instrument, make a cut along the longitudinal axis and proximate the middle of the tube starting from the distal end of the body and extending to a point about 4-5 mm from the enlarged head portion. This process will result in two elongate members 262. Additional elongate members are formed making additional cuts. For example, using surgical scissors cut down the middle a selected elongate member to a point about 4-5 mm away from the enlarged head portion. This process can be repeated on the other elongate member to create four elongate members. Additional elongate members can be formed by making additional longitudinal cuts.

Once the enlarged head and elongate members are formed, a neck portion 264 can be formed between the enlarged head 256 and the elongate members 262 (step 209). In various embodiments, the neck portion can be formed via ultrasonic welding (e.g., via a handheld welder). See FIG. 4I.

A skilled artisan can readily determine conditions and parameters suitable for ultrasonic welding. In an exemplary embodiment, for a body without any surface modification, the settings of the ultrasonic handheld welder can be applied as follows: 4 amplitudes and a weld time of about 0.25 seconds. Pinch the neck 264 proximate the head 256 (either with gloved hands or a grasping tool, such as tweezers) and introduce the body to the welder 266. Apply the head of the welder proximate the inside of the body where the opening of the tube-like structure 258 exists after the creation of the elongate members 262. Activate the welder to apply the amplitudes in order to seal off the neck area 264 of the body. Repeat this process until the neck area is completely sealed with minimal to no damage to the head 256. The resulting device will comprise a body 252 with an enlarged head portion 256, a neck portion 264 with a reduced cross-sectional area relative to the head 256, and a plurality of elongate members 262 extending outwardly from the neck 264. In the event that there is excessive damage to the body 252 (e.g., the head 256 can be easily pulled off or there are too many holes where the welder was applied), the device can be discarded and not subjected to tensile testing or used for implantation.

For a body with a surface modification (e.g., exposed to EDA, hydrolyzed, or loaded with a therapeutic agent), the welding parameters can be adjusted appropriately, e.g., in some embodiments, to 5 amplitudes and a weld time of about 1.0 seconds. The rest of the process is the same as that described above with respect to the unmodified body. The process is repeated until the neck area is completely sealed with minimal to no damage to the head 256.

Once the device is completed, it can be subjected to certain quality control procedures, such as measuring the device against a template to ensure that the desired measurements (e.g., diameter of the head, thickness of the head, length of the elongate members from the neck/weld, where the cut begins on the elongate members, thickness of each elongate member, width at the base of each elongate member, and the weight of each device) have been obtained. The device can be subjected to additional testing (e.g., tensile strength) as necessary and either further modified or prepared for implantation.

In some aspects, provided herein are methods of treating a patient having a fistula, using a fistula plug device described herein. As used herein, “treating” a patient having a fistula includes stabilizing or improving one or more conditions associated with the patient's fistula tract, e.g., by closing, filling, plugging, healing, shrinking, reducing the size of, or reducing symptoms associated with, the patient's fistula tract.

The diameter and length of the fistula plug can be sized to approximate the diameter and length of the patient's fistula tract. In exemplary embodiments, the fistula plug will fill all or a portion of the fistula tract following administration of the plug to the patient.

Virtually any type of fistula can be filled using the fistula plug devices described herein. For example, the fistula plug devices described herein can be used to treat a subject having a perianal fistula, a cryptoglandular anal fistula, a rectovaginal fistula, an enterocutaneous fistula, or an enteroatmospheric fistula. In some subjects, the fistula may be secondary to inflammatory bowel disease (IBD), e.g., Crohn's disease, ulcerative colitis, or indeterminate colitis. In some subjects, the fistula may be associated with chronic diarrhea, post-pregnancy complications, colitis, or rectal cancer. Fistula plug devices described herein can also be used to treat gastrointestinal fistulas, enterovesicular fistulas, and complex fistulas. Some fistulas have a single, primary opening, with a fistula tract or tunnel extending therefrom. Some fistulas additionally have one or more secondary opening(s) connected to the fistula tract. Some fistulas may have multiple tracts. Patients being treated for ulcerative colitis or indeterminate colitis may undergo an ileal pouch anal anastomosis (IPAA) procedure that generates an internal pouch. Patients undergoing an IPAA procedure have a higher risk for post-operative fistulae formation. Such fistulae may also be filled using the fistula plug devices described herein.

Accordingly, in some embodiments, provided herein are methods of treating, healing, or closing a fistula in a subject, comprising administering a fistula plug device described herein to a subject in need thereof. In some embodiments, the subject has a perianal fistula, a cryptoglandular anal fistula, a rectovaginal fistula, an enterocutaneous fistula, an enteroatmospheric fistula, a gastrointestinal fistula, an enterovesicular fistula, or a complex fistula. In some embodiments, the subject has a fistula located between the rectum and vagina, the large intestine and the surface of the skin, the stomach and the surface of the skin, the uterus and the peritoneal cavity, an artery and a vein, a bile duct and the surface of the skin, the cervix and the vagina, the neck and the throat, and/or in the nasal sinus cavity. The fistula plug device can be maintained in the fistula for a period of time sufficient for treating, healing, or closing the fistula.

In some embodiments, the subject is receiving concurrent treatment for a disorder to which the fistula is secondary, e.g., inflammatory bowel disease (IBD), e.g., Crohn's disease, ulcerative colitis, or indeterminate colitis. For example, in some embodiments, the subject can receive concurrent treatment with an antibody, or antigen binding portion thereof, that specifically binds α4β7 integrin. In some embodiments, the antibody, or antigen binding portion thereof, comprises a heavy chain variable region comprising a CDR1 of SEQ ID NO:2, a CDR2 of SEQ ID NO:3, and a CDR3 of SEQ ID NO:4, and/or a light chain variable region comprising a CDR1 of SEQ ID NO:6, a CDR2 of SEQ ID NO:7, and a CDR3 of SEQ ID NO:8. In some embodiments, the antibody, or antigen binding portion thereof, comprises a heavy chain variable region comprising SEQ ID NO:1, and/or a light chain variable region comprising SEQ ID NO:5. In some embodiments, the antibody that specifically binds α4β7 integrin is vedolizumab, or an antigen binding portion thereof. Vedolizumab is also known by the trade name ENTYVIO® (Millennium Pharmaceuticals, Inc.). Vedolizumab is a humanized monoclonal antibody that specifically binds to the α4β7 integrin, e.g., the α4β7 complex, and blocks the interaction of α4β7 integrin with mucosal addressin cell adhesion molecule-1 (MAdCAM-1) and fibronectin, and inhibits the migration of lymphocytes, e.g., CD4, CD8 or memory T-lymphocytes across the endothelium into inflamed gastrointestinal parenchymal tissue. The use of vedolizumab in patients with perianal fistulizing Crohn's disease is described, for example, in U.S. Patent Publication No. 2017/0327584A1, the entire contents of which are incorporated herein by reference.

The fistula plug device can be administered to the patient using standard surgical techniques. Any method resulting in implantation of the fistula plug in the fistula tract of a subject can be used to administer the device.

In some embodiments, the fistula plug can be inserted into the fistula tract through the primary opening. In other embodiments, the fistula plug can be inserted into the fistula tract through a secondary opening. In other embodiments, an opening to the fistula tract is created surgically, for insertion of the fistula plug.

Either end of the fistula plug device may be inserted first. In some embodiments, the fistula plug is inserted into the fistula tract using a gentle application of force. In other embodiments, the fistula plug may be attached to surgical sutures or string, and can be pulled into position within the fistula tract. In general, the fistula plug device is positioned such that the widest portion of the device is placed in the widest portion of the fistula tract.

The fistula tract may be washed and/or debrided prior to administration of the fistula plug device.

In some embodiments, the fistula plug device can be soaked in sterile saline prior to administration.

Following insertion, the fistula plug can be held in place using surgical sutures or stitches. In embodiments in which the fistula plug comprises a non-biodegradable polymer, it may be desirable to hold the fistula plug in place using non-biodegradable sutures. In some embodiments, the fistula plug is held in place using non-biodegradable sutures that comprise a nanofibrous polymer, e.g., a polymer of the same type present in the fistula plug.

The device can be maintained in the fistula tract of the subject for any period of time, e.g., a time sufficient for healing or partial healing of the fistula. For example, the fistula plug can be maintained in the fistula tract for a period of time sufficient for closure of one or more openings to the fistula tract, reduction of inflammation in the fistula tract, reduction of infection in the fistula tract, reduction in size of the fistula tract, and/or ingrowth of tissue into the fistula tract. In some embodiments, the fistula plug is not removed from the subject. In some embodiments, the fistula plug is maintained in the subject for 1 week or more, e.g., 1, 2, 3, 4, 5 weeks, etc. In some embodiments, the fistula plug is maintained in the subject for 1 month or more, e.g., 1, 2, 3, 4, 5, 6, 7, 8, 9, 10, 11, 12 months or more. In some embodiments, the fistula plug is maintained in the subject for 1 year or more, e.g., 1, 2, 3, 4, 5, 6, 7, 8, 9, 10 years or more. In some embodiments, the fistula plug is maintained in the subject indefinitely. In some embodiments, the fistula plug is permanently implanted in the subject. Preferably, the fistula plug device does not induce fibrosis at the site of insertion, and/or does not induce collagen deposition at the site of insertion. The fistula plug device can promote healing of the fistula tract by permitting ingrowth of tissue into the device and/or altering cellular dynamics at the site of insertion. In embodiments in which the device is loaded with a therapeutic agent, the device can also deliver the agent to the fistula tract, conferring additional benefit to the patient.

Also provided are kits comprising a fistula plug device, as disclosed herein. In some embodiments, the kits can comprise instructions for use of the device for treatment of a fistula. For example, the instructions may contain instructions for surgical implantation of the device, and/or instructions for loading the device with a therapeutic agent. In some embodiments the kit can contain non-biodegradable sutures for use implanting the device in a patient. In some embodiments, the non-biological sutures can comprise an electrospun nanofibrous polymer. In some embodiments, the kits can further comprise one or more surgical instruments for insertion of the device into a patient.

### Examples

The following examples are put forth so as to provide those of ordinary skill in the art with a description of how the devices and methods described herein may be used, made, and evaluated. The examples are intended to be purely exemplary and are not intended to in any way limit the scope of the invention.

### Example 1: Device Surface Modification

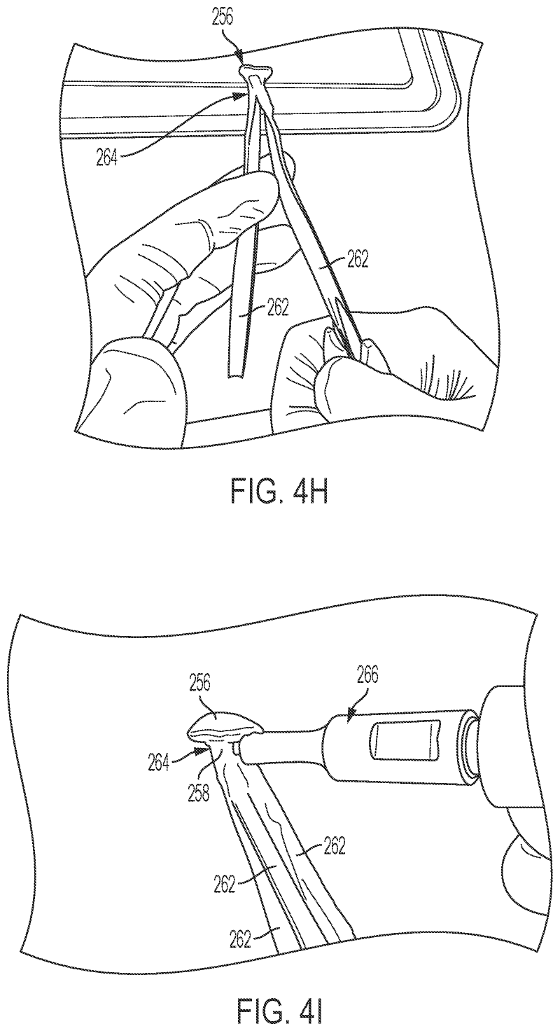

FIG. 5 depicts three fistula plugs 210a, 210b, 210c, where two of the plugs 210b, 210c have been subjected to a surface modification and the third plug is a control plug 210a. Plug 210b represents a HYD plug modified using sodium hydroxide to have a negative charge and bound to a positively charged dye (methylene blue) and shown as [symbol] . Plug 210c represents an EDA plug modified with liquid ethylenediamine to have a positive charge and bound to a negatively charged dye (acid red 1) and shown as [symbol] . Neither dye was bound to the control plug 210a. As can be seen, the surface modifications were effective based on the substantial and consistent coloring (shown as texturing in the figures) of plugs 210b, 210c.

### Example 2: Device Placement

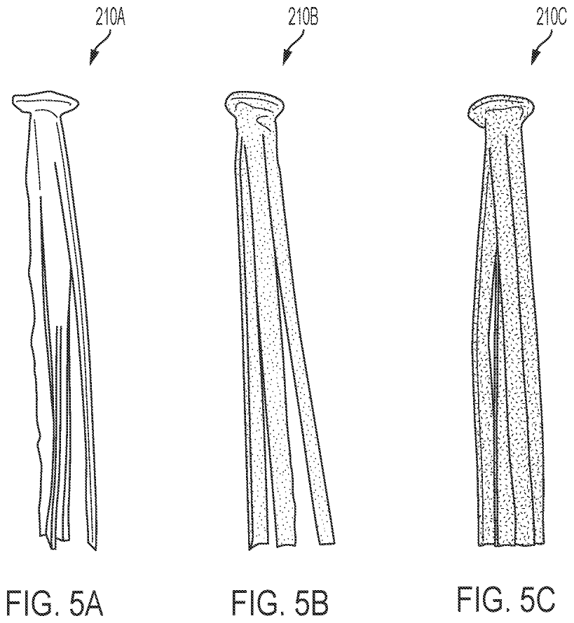

FIGS. 6A and 6B generically depict two possible applications for the devices (310 generally) described herein. FIG. 6A depicts a rectovaginal fistula 330, while FIG. 6B depicts an enterocutaneous fistula 332. The rectovaginal fistula 330 may occur as a result of Crohn's disease and will benefit from the implantation of a device 310a capable of delivering a cellular material to the site. The enterocutaneous fistula 332 may occur as a result of a complication from abdominal surgery and will also benefit from the implantation of a device 310b carrying a biological material to fortify the boundary between the intestines and the skin.

The particular device 310 to be used can be configured to fit a specific fistula site and to deliver a sustained level of a therapeutic agent directly to the site to, for example, alter the cellular dynamics at the site of trauma. In various embodiments, the device includes a therapeutic agent, such as, for example, a small molecule or an antibiotic, that can be incorporated into the electrospun fibers or otherwise captured by the scaffold-like structure of the device body. In some embodiments, the device can be soaked in the therapeutic agent prior to implantation or incorporated as part of the manufacturing process. The therapeutic agent can include, for example, an antimicrobial, an immunosuppressant, an anti-inflammatory, an analgesic, or cells, such as those disclosed herein.

### Example 3: Cell Loading

Generally, in some embodiments, the therapeutic agent includes allogenic adipose tissue-derived stromal stem cell, which can be incorporated into the device via soaking in a solution including the allogenic adipose tissue-derived stromal stem cell for about 30 to 60 minutes. The incubation times for attaching the cells will vary depending on the cells and materials used. Generally, the MSCs attached rapidly to all of the electrospun material surfaces in time-dependent fashion, where negatively charged nPET-PBD (HYD) showed comparable attachment to tissue culture plastic, while positively charged nPET-PBD (EDA) had a reduced level of MSC binding.

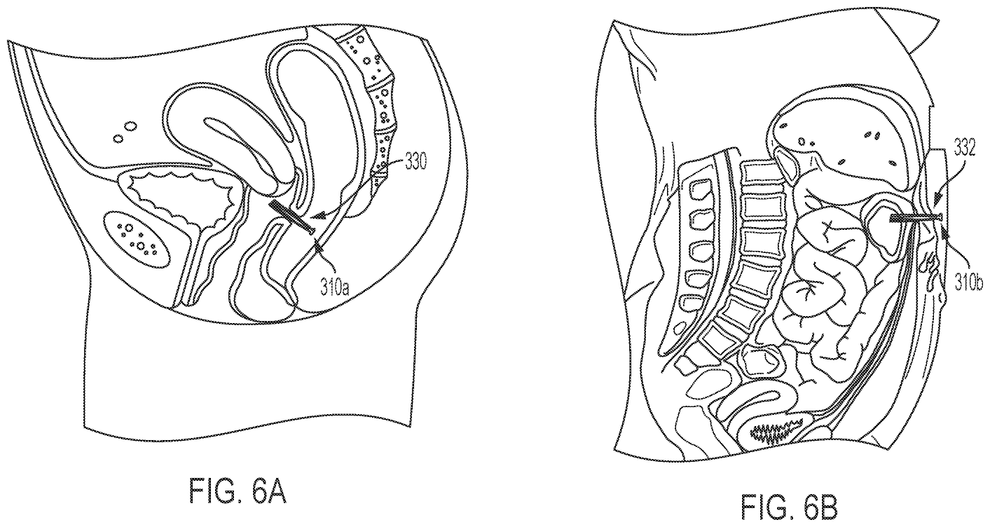

Human adipose derived Mesenchymal Stem Cells (MSCs) (passage 4) were seeded onto 16 mm diameter circular discs of electrospun polymer materials: nPET-PBT, nPET-PBT(EDA), and nPET-PBT(HYD), and normal tissue culture 24-well plates as controls at a density of 40,000 cells/well. Cells were incubated in 37° C. incubator under 5% CO2 condition in mesenchymal stem cell basal medium for adipose derived MSCs (ATCC® supplemented with 2% fetal bovine serum (FBS), 5 ng/mL rhFGF basic, 5 ng/mL rhFGF acidic, 5 ng/mL eh EGF, and 2.4 mM L-Alanyl-L-Glutamine). At 15, 30, and 60-minutes post seeding, the discs were transferred to fresh 24 well plates containing fresh culture medium, and incubated for another 90 minutes in the presence of Cell Counting Kit 8 (“CCK-8”, available from Dojindo Molecular Technologies, Inc.). As the standards, serially diluted MSCs were seeded into the normal tissue culture plate at a known density of 20,000, 10,000, 5,000, 2,500, 1,250, 625, 313, 156, and 78 cells/well and subjected to the same treatment as cells on the materials as above. The number of cells were back calculated based on the absorbance at 450 nm from each well. The graph presented in FIG. 7 illustrates the attachment times for MSCs to various electrospun materials.

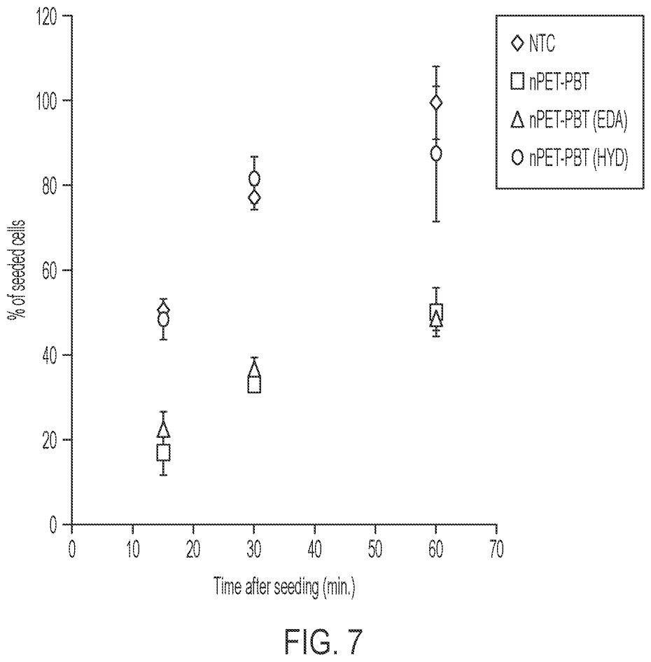

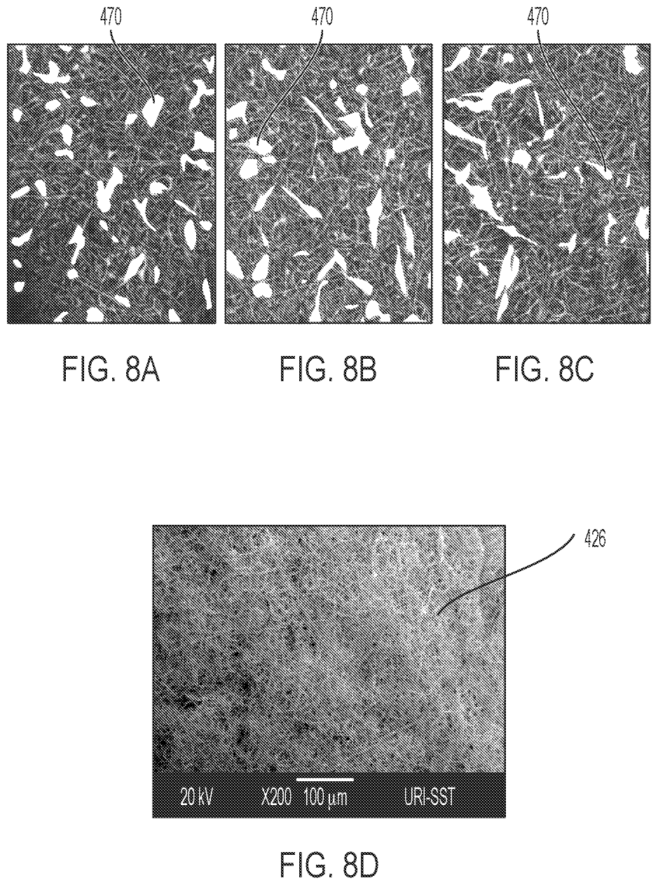

FIGS. 8A-8C depict MSCs growing on control (FIG. 8A), positively charged (EDA) (FIG. 8B), and negatively charged (HYD) (FIG. 8C) nPET-PBT electrospun polymer materials 426, and illustrates that the cells 470 adhered to the electrospun polymer 426 following seeding. The seeded cells 470 are generally shown in white. The following describes the protocol for seeding and staining the materials. Human adipose derived MSCs (passage 4) were stained with Celltracker Green™ (as available from Thermo Fisher Scientific) at a concentration of 5 μM for 30 minutes in a 37° C. incubator under 5% CO2 condition. After a phosphate-buffered saline (PBS) washing, the cells were trypsinized and harvested in the culture medium, mesenchymal stem cell basal medium for adipose derived MSCs (ATCC® supplemented with 2% FBS, 5 ng/mL rhFGF basic, 5 ng/mL rhFGF acidic, 5 ng/mL eh EGF, and 2.4 mM L-Alanyl-L-Glutamine). The cells 470 were seeded onto the presoaked 16 mm diameter circular discs of electrospun nanofibrous polymer materials 426: nPET-PBT, nPET-PBT(EDA), and nPET-PBT(HYD) at a density of 30,000 cells/well and were cultured in 37° C. incubator under 5% CO2 condition in the culture medium. Sixty-minutes post seeding, the medium was replaced with fresh medium, and cells were incubated overnight. The membranes with the cells were treated with 3.7% formaldehyde (SIGMA) for 15 minutes after 1 mL of PBS washing twice. 1 mL of 0.1% Triton X-100/PBS solution was added onto each of the materials and incubated for 15 minutes. An Image-iT® FX signal enhancer (as available from Thermo Fisher Scientific) was applied to the each of the materials and incubated for another 30 minutes at room temperature after PBS washing. Invitrogen™ Alexa Fluor™ 594 Phalloidin (as available from Thermo Fisher Scientific) staining was performed before observation with confocal laser scanning microscopy by incubating the materials with 5 μg/mL Phalloidin PBS solution containing 0.1% bovine serum albumin (BSA) for 60 minutes. FIG. 8D depicts an electrospun polymeric material 426 before seeding generally for reference purposes.

### Example 4: Drug Loading

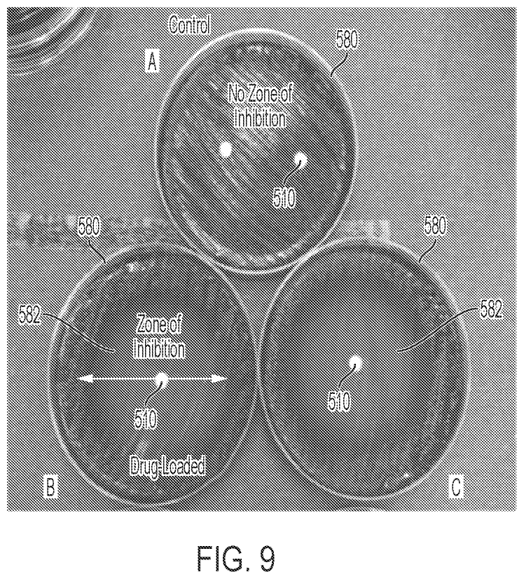

FIG. 9 depicts electrospun polymer materials manufactured as described herein and loaded with a small molecule antibiotic agent. Zones of inhibition 582 of bacterial growth are depicted surrounding non-drug loaded nPET-PBT plugs 510 (the white dots in the top well) and metronidazole-ciprofloxacin loaded plugs 510 (the white dots in the two bottom wells). Pre-selected amounts of Metronidazole and Ciprofloxacin were weighed and added to the PET-PBT polymer solution prior to electrospinning. As can be seen in the bottom wells 580, the drug was loaded into the nanofibrous polymer material and inhibited the growth of bacteria proximate the plugs (zones of inhibition 582 depicted as “clear” areas about the plugs 510). The following describes the protocol for preparing the materials. Generally, the protocol involved creating a bacterial lawn via streaking of blood agar plates, and embedding segments of the device.

A B. Fragilis KWIK-STIK™ pouch and an appropriate number of blood agar plates were brought to room temperature. Starting in the middle, blood agar plates were streaked rom one edge to the other, to cover the entire surface evenly.

Pieces of fistula plug material 510 were then embedded into the B fragilis-streaked plates 580. Non-drug loaded nPET-PBT plug segment (top plate), a Metronidazole loaded nPET-PBT plug (left bottom plate), and a Ciprofloxacin-Metronidazole nPET-PBT loaded plug (right bottom plate) were tested.

The plates 580 were incubated in a BD GasPak™ EZ Incubation Container (Becton Dickinson), with Anaerobe Container System sachets (Becton Dickinson) to create an anaerobic atmosphere. After 48 hours, the plates were examined for a clear ring around the sample (i.e., a zone of inhibition 582). No zone was apparent on plate A, indicating that there are no bactericidal properties provided by the fistula plug alone. A zone of inhibition 582 was apparent on plates B and C, indicating that the fistula plug 510 loaded with metronidazole-ciprofloxacin inhibited bacterial growth in the vicinity of the plug.

### Example 5: Device Implantation in a Rat Model

Electrospun nanofibrous fistula plugs 610 prepared in accordance with the methods described herein were administered to a subcutaneous tract 684 in a rat model, and maintained for a period of 30 days.

Briefly, a subcutaneous tract 684 was created in the rat dorsum such that the rat could not get access to the fistula plug 610 once implanted. After allowing the tract to form for 30 days (2 tracts per rat), rats were implanted with (i) no device (tract only); (ii) an unmodified nPET-PBT jelly configuration plug device, (iii) a nPET-PBT jelly configuration plug device negatively charged by treatment with sodium hydroxide (HYD), (iv) a nPET-PBT jelly configuration plug device positively charged by treatment with ethylenediamine (EDA); (v) a nPET-PBT jelly configuration plug device loaded with metronidazole); (vi) a nPU club configuration plug device; or (vii) a biodegradable plug device (control)(Gore Bio-A, Cook Medical) for an additional 30 days.

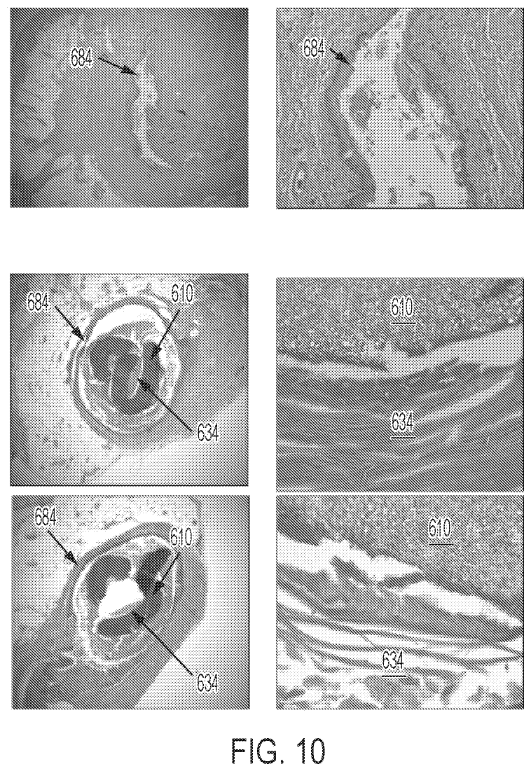

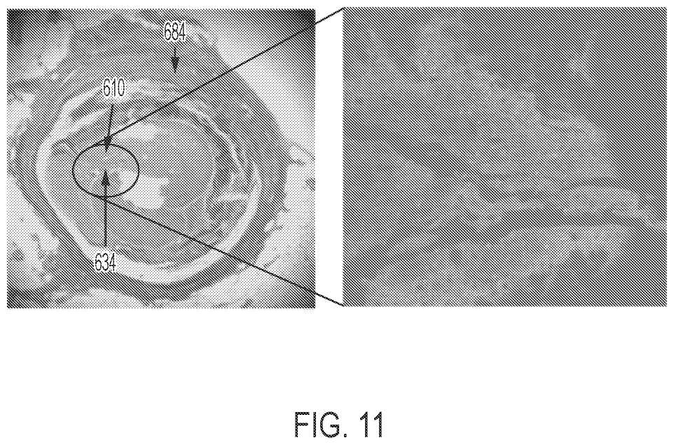

After 30 days, the devices were explanted, with all implants remaining in the tract as determined by gross observation. Results for the nPET-PBT plug are depicted in FIG. 10 (haemotoxylin and eosin (H&E) staining) and FIG. 11 (trichrome and immunostaining for a more detailed analysis (immunohistochemistry) of the jelly configuration plug device from the rat). Specifically, the top two photographs in FIG. 10 depict the tract 684 thirty days after suture removal, while the four bottom photographs depict the plugs 610 thirty days after suture removal. The photographs of FIG. 11 depict the interface between the tissue 634 and the plug 610 that is visible after application of the trichrome (Masson's) and immunostaining with CD-31-fuscia, collagen type III-green, and DAPI blue, highlighted in the right photograph.

Histologically, the HYD-jelly configuration had excellent tissue ingrowth, which appeared similar to ingrowth observed into these devices when implanted in the porcine model (discussed below). The metronidazole-loaded jelly configuration had healing similar to unmodified jelly configurations in that there is cellular penetration into the outer surface of the device. The nPU club configuration had less cellular penetration than the nPET-PBT jelly configuration, but still appeared to have some cellular penetration. For the control (Cook) device, there was limited cellular healing within the device in several devices examined.

### Example 6: Device Implantation in a Pig Model

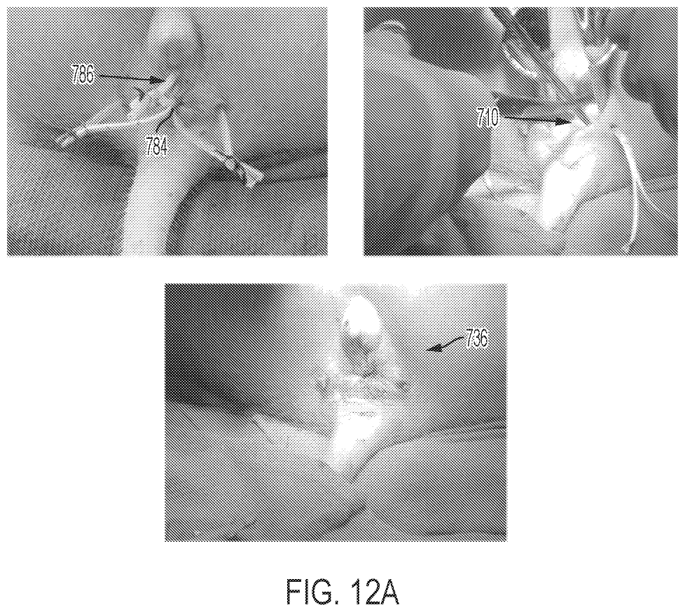

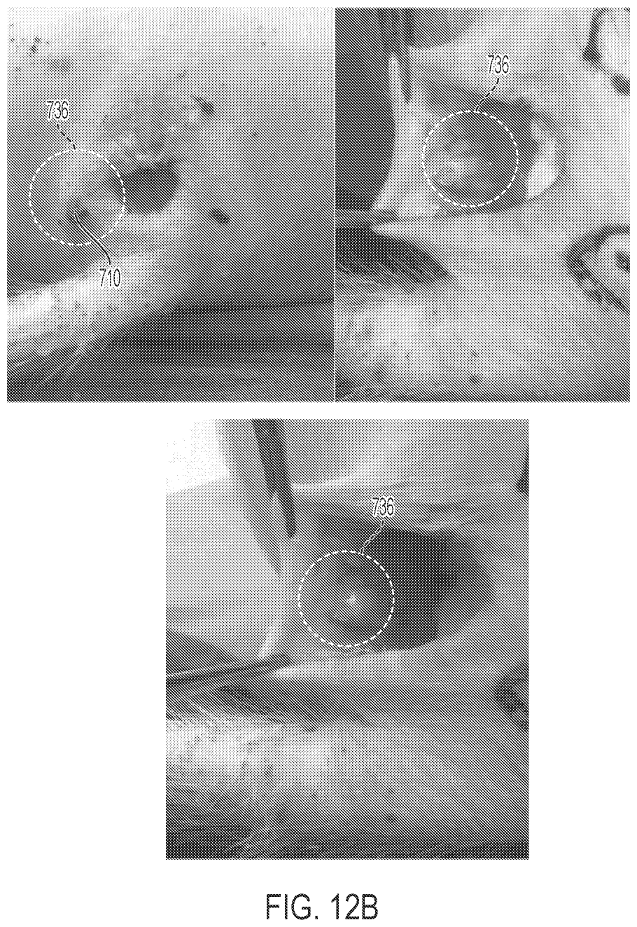

Electrospun nanofibrous fistula plugs 710 prepared in accordance with the methods described herein were then tested in a pig fistula model. For the porcine study, three (3) independent tracts 784 were created by inserting 10 French silicone drains 786 with a trocar as far back from the sphincter muscle as possible for 30 days. This resulted in a slightly longer tract 784. After 30 days, the drains 786 were removed and the tracts 784 were implanted with (i) no device (tract only); (ii) an unmodified nPET-PBT jelly configuration plug device, (iii) a nPET-PBT jelly configuration plug device negatively charged by treatment with sodium hydroxide (HYD), (iv) a nPET-PBT jelly configuration plug device positively charged by treatment with ethylenediamine (EDA); (v) a jelly configuration plug device loaded with metronidazole); (vi) a nPU club configuration plug device; or (vii) a biodegradable plug device 713 (control, Cook Medical) for an additional 30 days. In all cases, a small incision was made through the skin between the exit site on the skin and the mucosa. The plug 710 was inserted and anchored through the incision using a Mersilene® suture (available from J&J Medical Devices). The mucosa side was anchored using a 3-0 PDS® II suture (available from J&J Medical Devices). The photographs of FIG. 12A show the plugs at the time of insertion.

FIG. 12B is a collection of photographs of the tracts 784 in the skin in the porcine fistula model after 30 days of plug implantation. The exit site 736 of the jelly configuration device, indicated by the dashed line (top left photograph), showed excellent healing and incorporation. The top right and bottom photographs show the entrance tract on the mucosa portion in the porcine fistula model after 30 days of plug implantation, where the entrance site of the jelly configuration device 736, indicated by the dashed line, shows the head of the device 710 fully incorporated into the mucosal tissue.

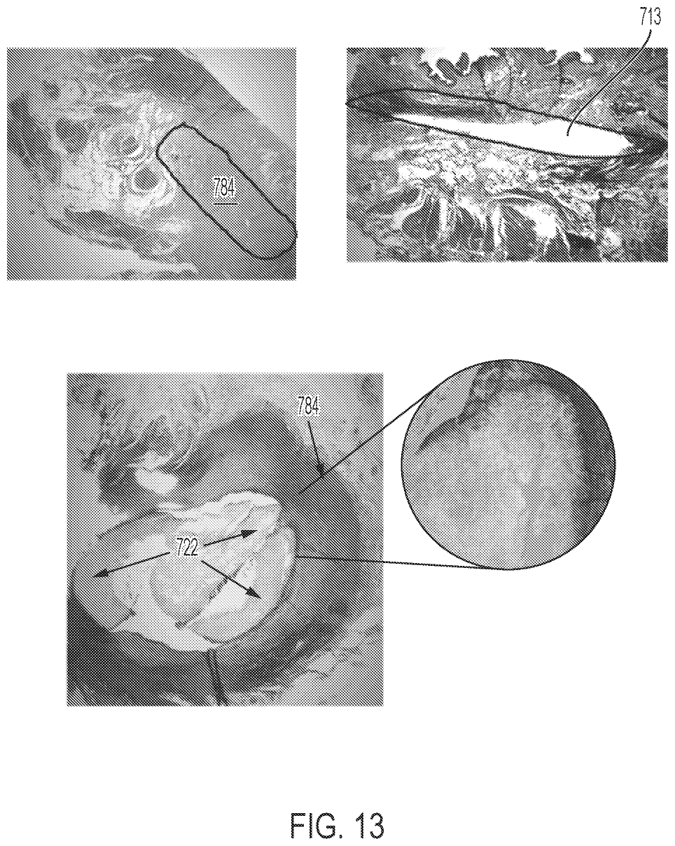

After 30 days, the devices were explanted. Histological assessment of the 30-day nPET-PBT jelly configuration showed that the devices had tissue ingrowth into the elongate members of the jelly configuration, with greater ingrowth focused on the outer surfaces of the devices. There were also cells infiltrating into the inner portions of the devices, but not to the same level. These results were similar to what was observed with the rat dorsal subcutaneous implants described in the previous example. Trichrome staining showed the presence of the tract, indicated by intense collagen staining. There was minimal collagen deposition around or near the jelly configuration as generally shown in the photographs of FIG. 13. Specifically, the top left photograph depicts a control tract 784 without a device implanted therein and where a collagen deposit has formed after thirty days, and the top right photograph depicts implantation of the control device 713 where no tissue infiltration occurred resulting in the loss of the plug. The bottom photograph depicts the tissue growth that penetrated into the tract area between the device elongate members 722 and the tract walls representing deep cellular penetration similar to that obtained in the rat studies.

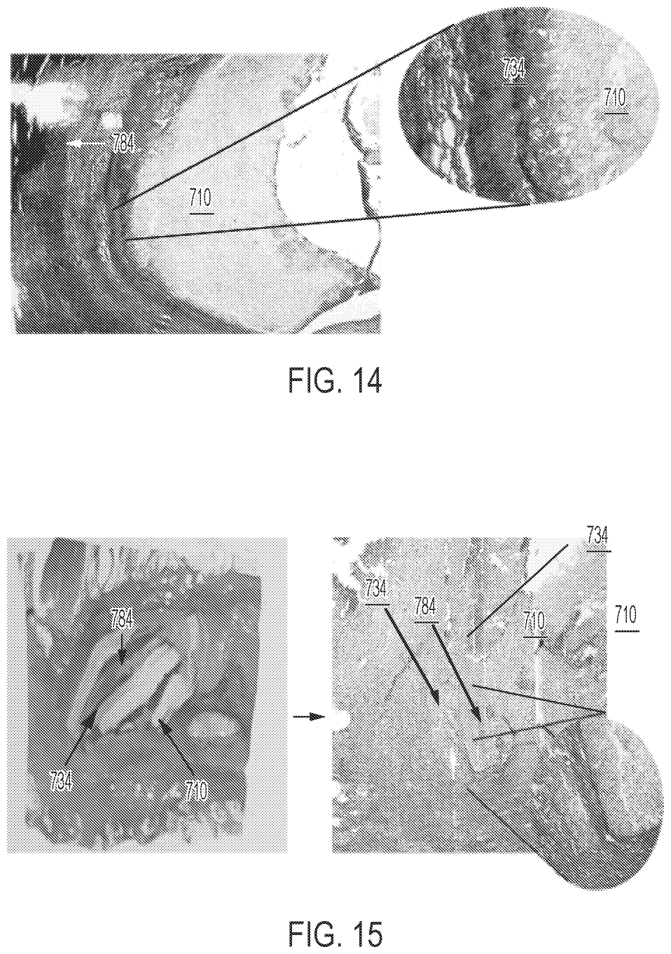

One anionic and one cationic nPET-PBT jelly configuration were explanted from the porcine model at 16 days. Histological assessment showed both devices having tissue ingrowth, which appeared to happen at a faster rate than the nPET-PBT control jelly configuration at 30 days. Cells also appeared to penetrate deeper into the elongate members. Trichrome staining showed similar results to the 30-day explants in that the tract was intact and there was minimal collagen staining around the devices (see FIGS. 14 and 15).

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives.

Furthermore, those skilled in the art should appreciate that the parameters and configurations described herein are exemplary and that actual parameters and/or configurations will depend on the specific application in which the systems and techniques of the invention are used. Those skilled in the art should also recognize or be able to ascertain, using no more than routine experimentation, equivalents to the specific embodiments of the invention. It is, therefore, to be understood that the embodiments described herein are presented by way of example only and that, within the scope of any appended claims and equivalents thereto; the invention may be practiced other than as specifically described.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

SEQUENCE TABLE

| SEQ | | |
|---|---|---|
| ID | | |
| NO: | DESCRIPTION | SEQUENCE |
| 1 | Heavy chain | QVQLVQSGAEVKKPGASVK |
| | (HC) variable | VSCKGSGYTFTSYWMHWVR |
| | region | QAPGQRLEWIGEIDPSESN |
| | (amino acid) | TNYNQKFKGRVTLTVDISA |
| | | STAYMELSSLRSEDTAVYY |
| | | CARGGYDGWDYAIDYWGQG |
| | | TLVTVSS |
| 2 | HC CDR1 | SYWMH |
| | (amino acid) | |
| 3 | HC CDR2 | EIDPSESNTNYNQKFKG |
| | (amino acid) | |
| 4 | HC CDR3 | GGYDGWDYAIDY |
| | (amino acid) | |
| 5 | Light chain (LC) | DVVMTQSPLSLPVTPGEPA |
| | variable region | SISCRSSQSLAKSYGNTYL |
| | (amino acid) | SWYLQKPGQSPQLLIYGIS |
| | | NRFSGVPDRFSGSGSGTDF |
| | | TLKISRVEAEDVGVYYCLQ |
| | | GTHQPYTFGQGTKVEIK |
| 6 | LC CDR1 | RSSQSLAKSYGNTYLS |
| | (amino acid) | |
| 7 | LC CDR2 | GISNRFS |
| | (amino acid) | |
| 8 | LC CDR3 | LQGTHQPYT |
| | (amino acid) | |

1. A device for implantation into a fistula tract within a subject, wherein the fistula tract has a primary opening in a wall of a bodily structure and a tract extending from the primary opening, the device comprising:
a unitary body configured to fill at least a portion of the fistula tract, wherein the body comprises:
an enlarged portion located at a first end of the body; and
a tube-like structure extending from an area proximate the enlarged portion and terminating at a second end of the body, wherein the enlarged portion has a cross-sectional dimension equal to or greater than a cross-sectional dimension of the tube-like structure relative to a longitudinal axis of the body.

2. The device of claim 1, wherein at least a portion of the tube-like structure is tapered towards the second end of the body.

3. The device of claim 1, wherein at least a portion of the tube-like structure is split into a plurality of elongate members.

4-9. (canceled)

10. The device of claim 1, wherein the fistula tract is a perianal fistula, an enterocutaneous fistula or a rectovaginal fistula.

11. The device of claim 1, wherein the body comprises one or more nanofibrous synthetic materials.

12-24. (canceled)

25. The device of claim 1, wherein the device comprises an electrospun material that has a therapeutic agent incorporated into the nanofibers.

26. The device of claim 25, wherein the therapeutic agent is selected from the group consisting of a small molecule, an antimicrobial agent, an anti-inflammatory agent, immunosuppressive agent, or an analgesic agent.

27-35. (canceled)

36. The device of claim 1, wherein the device further comprises cells selected from the group consisting of allogeneic stem cells, autologous stem cells, mesenchymal stem cells, allogenic adipose tissue-derived stromal stem cells, or a combination thereof.

37-39. (canceled)

40. A device for implantation into a fistula tract within a subject, wherein the fistula tract has a primary opening in a wall of a bodily structure and a tract extending from the primary opening, the device comprising:
a body configured to fill at least a portion of the fistula tract and at least partially formed from an electrospun nanofibrous polymer, wherein the body comprises:
an enlarged portion located at a first end of the body; and
a tube-like structure extending from the enlarged portion and terminating at a second end of the body, wherein the enlarged portion has a cross-sectional dimension equal to or greater than a cross-sectional dimension of the tube-like structure relative to a longitudinal axis of the body.

41. The device of claim 40, wherein the fistula tract is a perianal fistula, an enterocutaneous fistula or a rectovaginal fistula.

42-64. (canceled)

65. The device of claim 40, wherein the body is a unitary structure.

66. The device of claim 40, wherein the body further comprises a neck located between the enlarged portion and the tube-like structure, the neck comprising a reduced cross-sectional dimension relative to the cross-sectional dimensions of the enlarged portion and the tube-like structure.

67-72. (canceled)

73. The device of claim 40, wherein the second end is configured for insertion into the primary opening and the first end is configured to fill all or a portion of the fistula tract.

74. The device of claim 40, wherein the first end is configured to fill the primary opening.

75. A method of manufacturing a device for implantation into a tract within a subject such as a fistula, which has a primary opening in a wall of a bodily structure and a tract extending from the primary opening, the method comprising:
preparing a polymeric solution;
loading the solution into an electrospinning unit;
perfusing said polymeric solution at a defined rate into an injection needle port that has a defined voltage applied to the needle;
collecting the resulting nanofibers emanating from the needle port onto a rotating collector in the form of a mandrel having a fluted portion to create a unitary tube-like electro-spun body with a fluted portion;
performing a post-treatment process, such as vacuum heating or solvent extraction, to remove residual solvent;
removing the electro-spun body from the mandrel;
manipulating the fluted portion by pulling the fluted end downward to form an enlarged portion relative to the rest of the body, wherein the enlarged portion has a cross-sectional dimension larger than a cross-section dimension of the rest of the body;
flattening the rest of the body to form a substantially planar structure extending from the elongate portion;
splitting the planar structure along a longitudinal axis of the body to form a plurality of elongate members; and
forming a neck region between the enlarged portion and the elongate members by sealing a portion of the tube-like body proximate to the enlarged portion, wherein the neck region has a cross-sectional dimension less that the cross-sectional dimensions of the enlarged portion and the rest of the body.

76-80. (canceled)

81. The method of claim 75, wherein the polymeric solution has at least one therapeutic agent added into the solution prior to electrospinning, wherein the therapeutic agent is selected from the group consisting of a small molecule, an antimicrobial agent, an anti-inflammatory agent, immunosuppressive agent, or an analgesic agent.

82-91. (canceled)

92. The method of claim 75 further comprising performing a surface modification on the body, such as an anionic or cationic surface modification.

93. The method of claim 75, wherein the step of forming the neck region comprises ultrasonically welding the portion of the tube-like body proximate to the enlarged portion.

94. A kit comprising a device for implantation in a fistula in accordance with claim 1.

95. The kit of claim 94 further comprising a non-biodegradable suture.

96. The kit of claim 95, wherein the non-biodegradable suture comprises an electrospun material.

97. (canceled)

98. A method of treating a patient having a fistula that has a primary opening in the wall of a bodily structure, and a fistula tract extending from the primary opening, the method comprising insertion of the device of claim 1 into the fistula tract through the primary opening.

99. The method of claim 98, wherein the device fills at least a portion of the fistula tract.

100. The method of claim 98, wherein the patient has a perianal fistula, a rectovaginal fistula, an enterocutaneous fistula, an enteroatmospheric fistula, or a cryptoglandular anal fistula.

101. (canceled)

102. The method of claim 98, wherein the patient has Crohn's disease or ulcerative colitis.

103-108. (canceled)

109. The method of claim 98, wherein the device permits ingrowth of tissue into the device.

110-111. (canceled)
